(19) United States
(12) Patent Application Publication
(10) Pub. No.: US 2023/0357695 A1
(43) Pub. Date: Nov. 9, 2023

(54) SYSTEMS AND METHODS FOR HOLDING AN INSTRUMENT IN A PETRI DISH

(71) Applicant: Harvard Bioscience Inc., Holliston, MA (US)

(72) Inventor: Tyler Robert Eisner, Blackstone, MA (US)

(21) Appl. No.: 18/143,433

(22) Filed: May 4, 2023

Related U.S. Application Data
(60) Provisional application No. 63/338,259, filed on May 4, 2022.

(51) Int. Cl. C12M 1/42; B01L 9/00; C12M 1/22; C12M 3/00

(52) U.S. Cl. CPC C12M 35/02; B01L 9/527; C12M 23/10; C12M 23/48; B01L 2300/045; B01L 2300/0803; B01L 2200/025; B01L 2300/0645

(57) ABSTRACT

Some systems, devices and methods detailed herein provide a restraint device for holding a microslide or another slide instrument in an operative position in a petri dish.

### CROSS-REFERENCE TO RELATED APPLICATIONS

This application claims the benefit of U.S. Provisional Application Ser. No. 63/338,259, filed May 4, 2022. The disclosure of the prior application is considered part of (and is incorporated by reference in) the disclosure of this application.

### TECHNICAL FIELD

This document describes systems, devices, and methods for retaining a slide instrument used, for example, during electroporation or electrofusion. Particular examples described herein provide improved positioning and releasable restraint of a microslide, a microscope slide instrument, or an observation device arranged within a petri dish.

### BACKGROUND

Microslides, microscope slides, and other lab observation devices are used in a wide variety of applications in research and medical industries. Such devices can be used with or without a petri dish to hold one or more samples for treatments, observation, and procedures in a lab environment. For example, microslides are often seated within a transparent petri dish during electroporation or electrofusion procedures for purposes of observing cell fusion, cell nuclear transfer, plant protoplast fusion, and embryo manipulation processes. Such devices can be fit on a microscope stage for observation (under magnification) by a user. When microslides, microscope slides, and other lab observation devices are unrestrained in a petri dish, these devices are subject to unintended or inadvertent movements relative to the walls of the petri dish. Alternatively, some petri dish configurations include an acrylic plate bonded to a sidewall of the petri dish so that a microslide inserted into the petri dish will abut against a flat wall of the acrylic plate, but such a configuration is prone to failure at the adhesion point of the acrylic plate upon the petri dish sidewall, which may lead to a loss of a sample, inconsistent processing or observation of the sample, and a loss of equipment.

### SUMMARY

This document describes systems, devices, and methods for retaining a microslide, a microscope slide instrument, or another instrument in an observation container. In particular implementations, the systems, devices, and methods described herein can include a removable tool that achieves improved capturing and releasable coupling of a microscope slide, instrument, or measurement device within the observation container (e.g., petri dish in particular embodiments) sized to fit on a microscope stage for observation. Some embodiments of systems and methods detailed herein include providing improved capturing, positioning, and (optionally) removably restraining of a microslide, instrument, or measurement device within a petri dish used with an electroporation system.

Among other benefits, some systems and methods described herein can provide a more efficient and robust approach to capturing, positioning, (optionally) removably restraining a microslide, instrument, or measurement device within a petri dish. Additionally, some embodiments described in more detail below can achieve a removable, reusable solution that is advantageously usable in various petri dish sizes and is readily adjustable to engage with different sizes and shapes of microslides, instruments, or measurement devices.

In some embodiments, a restraint device for retaining a slide instrument in a petri dish is provided. The restraint device also includes a spring bias component having one or more arms, each arm having one or more elbows; and a housing connected to the spring bias component, the housing having a base that defines a recess to releasably mate with at least a portion of a slide instrument; where the one or more arms of spring bias component are shaped to elastically deform in response to engagement with a wall of a petri dish so that the spring bias component generates a retainer force applied from the base and toward the recess.

A number of embodiments include a restraint device where a portion of the spring bias component is integrated in the base of the housing. The base of the housing defines a slot that receives a portion of the spring bias component to connect the spring bias component to the housing. The recess of the base extends longitudinally away from a rear face of the base and is configured to slidably receive the microslide at a front face of the base. The spring bias component has one or more handles that extend from a free end of each arm. The spring bias component may include a spring wire. The spring bias component may include an elastic material. The base of the housing has a slot that receives a portion of the spring bias component to connect the spring bias component to the housing. The restraint device is symmetric about a longitudinal plane that extends along the recess of the base. The spring bias component has one or more handles that extend from an end of each arm. The spring bias component may include a spring wire. The retainer force applied from the base is longitudinally toward the microslide when the microslide is releasably mated with the base. A portion of the spring bias component is integrated in the base of the housing. The spring bias component may include an elastic material.

Particular implementations can, in certain instances, realize one or more of the following advantages. The systems, devices, and methods described herein provide a more secure and consistent approach to holding microslides, instruments, and measurement devices within a petri dish that reduces the likelihood that a sample may spill off of the microslide and maintains the original position of the microslides, instruments, and/or measurement device. Additionally, the presently described systems, devices, and methods offer a removable, reusable solution that can be used in various petri dish sizes and can be adaptable to work with different sizes and shapes of microslides, instruments, or measurement devices.

The details of one or more implementations are set forth in the accompanying drawings and the description below. Other features, objects, and advantages will be apparent from the description and drawings, and from the claims.

### DESCRIPTION OF DRAWINGS

FIG. 1 shows a perspective view of an example electroporation system and components, consistent with embodiments of this disclosure.

FIG. 2 shows a perspective view of an example restraint device in a petri dish of the system of FIG. 1.

FIG. 3 shows a top view of the restraint device and petri dish of FIG. 2.

FIG. 4 shows a bottom view of the restraint device and petri dish of FIG. 2.

FIG. 5 shows a top perspective view of the restraint device of FIG. 2.

FIG. 6 shows a bottom perspective view of the restraint device of FIG. 2.

FIG. 7 shows a top view of the restraint device and petri dish of FIG. 2.

FIG. 8 shows a top view of another restraint device in another petri dish, consistent with some embodiments of this disclosure.

Like reference symbols in the various drawings indicate like elements.

### DETAILED DESCRIPTION

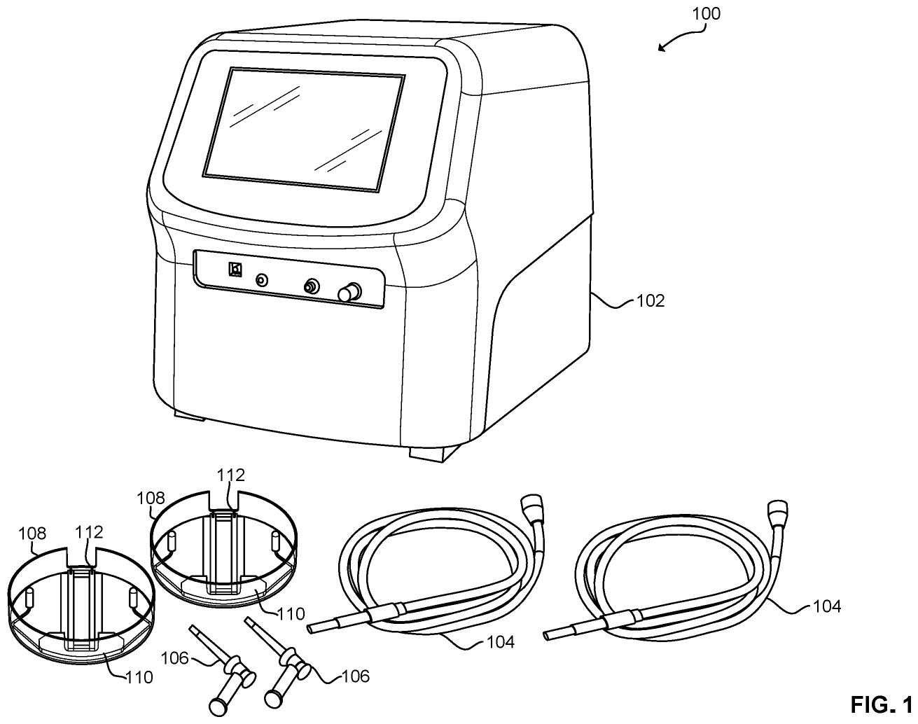

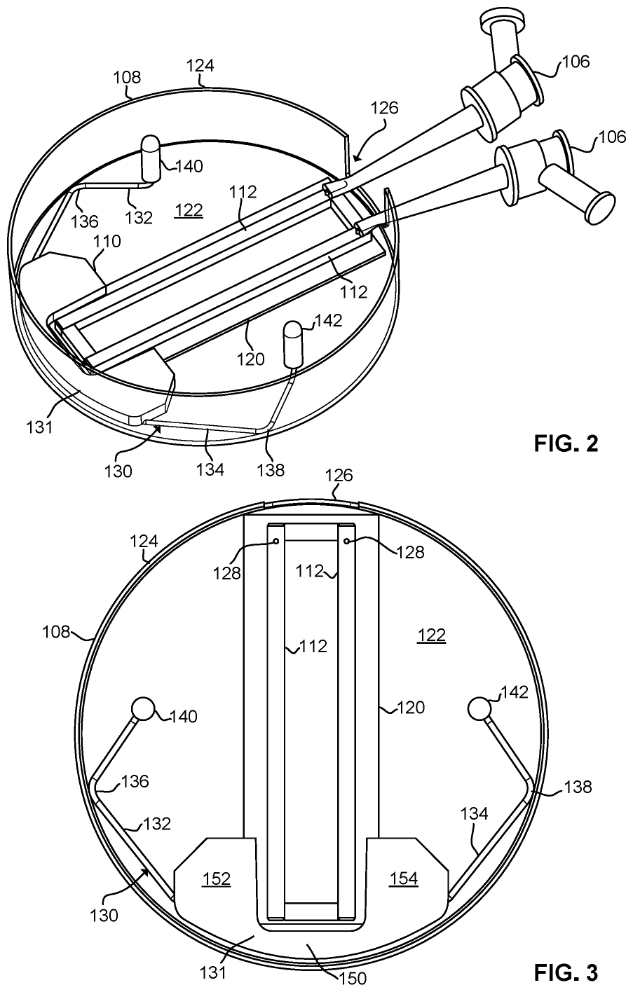

Referring to FIG. 1, some embodiments of an electroporation system 100 can include an electroporation machine 102, electroporation leads 104, electroporation connectors 106, one or more petri dishes 108 each containing a respective microslides 120 engaged with a restraint device 110 (FIG. 2). The electroporation system 100 can apply controlled electrical pulses to living cells (e.g., samples held in the petri dishes 108) in order to permeabilize the cell membrane for the purposes of transfection or transformation.

The electrical pulses are delivered to a pair of electrodes 112 by a pulse generator of the electroporation machine 102 via electroporation leads 104 that are connected to the pair of electrodes 112 via the electroporation connectors 106. In use, the electrical pulses at the electrodes 112 induce a transmembrane potential which causes the reversible breakdown of the cellular membrane, which can be observed on the microslide 120 (FIG. 2) under magnification. This action results in the formation of pores that allow molecules, such as DNA, proteins or antibodies, to enter the cell. Due to electroporation's reproducibility, high efficiency, and low toxicity, electroporation facilitates the introduction of a variety of molecules into cells such as mammalian, bacterial, yeast, plant, among others.

The electroporation system 100 is configured for electrofusion and electroporation. The electroporation system 100 incorporates both AC and DC square wave pulsing capabilities to allow for plant protoplast fusion, embryo manipulation and mammalian cell transfections. Cells on the microslide 120 (FIG. 2) can be manipulated under a microscope using the petri dish 108 (which contains a respective microslide 120 and restraint device 110 therein). The microslides, petri dishes 108, and electrodes 112 deliver a gentle, low intensity, high frequency AC pulse to align the cells of the sample at the microslide while the DC square wave pulse fuses the cells for higher fusion rates when compared to other chemical methods. The electroporation system 100 can function as the highly efficient AC/DC electrofusion system or to transfect a variety of mammalian cells, including direct gene delivery into oocytes or in vivo tissues by simply turning off the AC features and utilizing the DC square wave pulsing capabilities.

Referring now to FIGS. 2-4, some embodiments of the system 100 include the restraint device 110 that is configured to releasably retain a microslide 120 in an operative position within the petri dish 108. In the depicted embodiment, the petri dish 108 has a base plate 122 and a wall 124 that vertically extends from the base plate 122 around a circumference of the base plate 122. The wall 124 can include a wall opening 126 in a portion of the wall 124 that reduces the height of the wall 124 in the wall opening 126.

The electrodes 112 include a pair of electrodes in this embodiment, and other embodiments can include one or more electrodes based on the application (e.g., electroporation and/or electrofusion). The pair of electrodes 112 can be integrated, embedded, attached, or otherwise connected to the microslide 120. The pair of electrodes 112 can include two bars that extend parallel to each other across a portion of the microslide 120. The pair of electrodes 112 can be aligned with the wall opening 126, and can include electrode connectors 128 at the end of each of the electrodes. The electrode connectors 128 can be an extension of each of the pair of electrodes 112 that is configured for connection to the electroporation connectors 106. The electroporation connectors 106 can connect to the electrodes 112 via the electrode connectors 128 through the wall opening 126. While in some aspects the electrodes 112 are connected to the microslide 120, the electrodes 112 can be integrated, embedded, attached, or otherwise connected to the base plate 122.

The restraint device 110 can removably engage with the microslide 120 in the petri dish 108 to provide a secure fixation during use and to provide removability of the microslide 120 from the petri dish 108. The microslide 120 can be rectangular in shape and includes a major upper surface to receive one or more samples upon the microslide 120. In some implementations, the sample can be added to the surface of the microslide 120 before the microslide 120 is inserted into the petri dish 108. In other implementations, the sample can be added to the surface of the microslide 120 after the microslide 120 has been inserted into the petri dish 108 and engaged by the restraint device 110. As shown in FIGS. 2-4, the microslide 120 can be held in an operative position by the restraint device 110 while the restraint device 110 avoids interference with the field of view toward the sample on the major surface of the microslide 120.

Both the restraint device 110 and the microslide 120 can be entirely removable from the petri dish 108, for example, to achieve reuse of the restraint device 110, the microslide 120, or both within a second petri dish. The restraint device 110 includes at least one spring bias component 130 and an housing 131. The spring bias component 130 can include one or more arms. For example, the spring bias component 130 can include a first arm 132 and a second arm 134, which in this embodiment are flexible arms that extend from opposing lateral sides of the housing 131. The first arm 132 can include a first elbow 136 between two longitudinally straight arm portions, and the second arm 134 can include a second elbow 138 between two longitudinally straight arm portions. The first elbow 136 of the flexible first arm 132 and the second elbow 138 of the second arm 134 may be biased outwardly away from one another, and when seated in the petri dish 108, the elbows 136 and 138 press outwardly against an interior face of the wall 124 of the petri dish 108. The spring bias first arm and second arm may comprise a spring metal wire to facilitate elastic deformation of the spring bias arms 132 and 134 relative to the housing 131. Optionally, the spring bias component (including both arms 132 and 134) is formed from a single wire structure arranged in the depicted shape and secured to the housing 131. The flexibility and strength of the spring bias component 130 can urge the housing 131 toward the microslide 120 and thereby hold the microslide 120 within the petri dish 108 at the operative position.

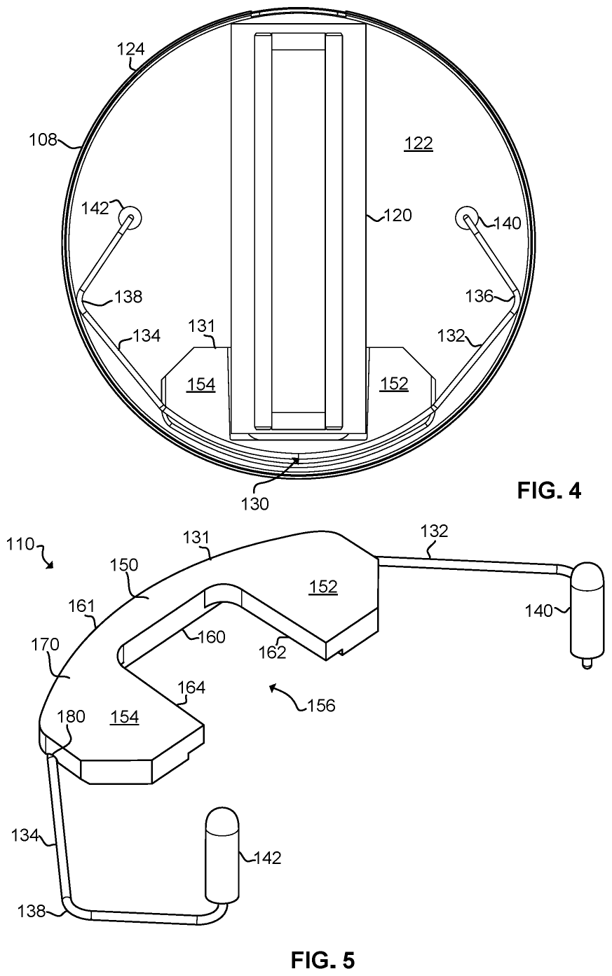

In some aspects, the first elbow 136 and the second elbow can define angles that are between 30 degrees and 150 degrees, including about 90 degrees in the example depicted in FIG. 3. As such, a proximal portion of the first arm 132 can extend away from the housing 131 and towards the wall 124 to join with the first elbow 136, and a distal portion of first arm 132 extends away from the first elbow 136 at an angle (e.g., about 90 degrees in this example) to extend away from the wall 124 and toward a free end spaced inwardly from the wall 124 (the free end may optionally include a handle 140, as detailed below). Similarly, a proximal portion of the second arm 134 can extend away from the housing 131 (outwardly away from the first arm 132) and towards the wall 124 to join with the second elbow 138, and a distal portion of second arm 134 extends away from the second elbow 138 at an angle (e.g., about 90 degrees in this example) to extend away from the wall 124 and toward a free end spaced inwardly from the wall 124 (the free end may optionally include a handle 140, as detailed below.) In some aspects, the first elbow 136 and the second elbow 138 can have equal angles, and the first arm 132 and the second arm 134 to be symmetrical on either side of the housing 131. For example, as depicted in FIGS. 3-4, the restraint device 110 may be symmetric about a longitudinal axis that bisects the recess 156 and extends parallel to the major surface of the microslide 120. In other aspects, the first elbow 136 and the second elbow 138 can have non-equal angles that cause the first arm 132 and the second arm 134 to be asymmetrical on either side of the housing 131.

Still referring to FIGS. 2-4, the spring bias component 130 can include one or more handles spaced apart from the housing for grasping by a user. For example, the spring bias component 130 includes a first handle 140 at the end of the first arm 132 and a second handle 142 at the end of the second arm 134. The first handle 140 and the second handle 142 can extend vertically from the ends of each the first arm 132 and the second arm 134. The first handle 140 and the second handle 142 can provide surfaces for a user to grip the restraint device 110 and adjust the relative positions of the first and second arms 132 and 134. For example the handles 140 and 142 can be engaged by a user fingers and pinched toward one another, thereby urging the elbows 136 and 138 away from the wall 124 of the petri dish 108 for simplified removal of the restraint device 110 from the petri dish 108 (or position adjustment within the petri dish 108). Likewise, upon release of the handles 140 and 142 from the user's grasp, the arms 132 and 134 are spring biased outwardly away from one another to thereby urge the elbows 138 and 138 into frictional engagement with the wall 124 of the petri dish 108. As previously described, the elbows 136 and 138 press against the curved wall 124 and urge the housing 131 toward the microslide 120, which is restrained in its position between the housing 131 at one end and the wall 124 at the opposing end.

In the depicted embodiment, the housing 131 of the restraint device 110 is connected to a central region of the spring bias component 130 (e.g., between the arms 132 and 134 that extend from opposing sides of the housing 131). For example, the housing 131 can include a base 150 that is connected to the spring bias component 130. The base 150 can connect a right side 152 and a left side 154 of the housing 131. The base 150 can extend between a base portion of each of the right side 152 and the left side 154. The right side 152 and the left side 154 extend away from the base 150 around an housing recess 156 (see e.g., FIG. 5) that is configured to slidably receive the microslide 120 between the right side 152 and the left side 154.

As shown in FIG. 4, for example, the petri dish 108 can be transparent such that the restraint device 110 and the microslide 120 are visible through the base plate 122. The spring bias component 130 can extend through the housing 131 and follow the shape of the housing 131. For example, as depicted in FIG. 4, the first arm 132 of the spring bias component 130 extends from a first side 152 of the housing 131 and the second arm 134 extends from a second side 154 (opposite from the first side 152). In some aspects, the first arm 132 and the second arm 134 can be a unitary structure (such as a single wire in this example) that extends from the first handle 140 to the second handle 142. The unitary structure can include angles and turns such as the first elbow 136 and the second elbow 138, and comprise a spring metal, nitinol, or a molded polymer configured to elastically flex as described herein.

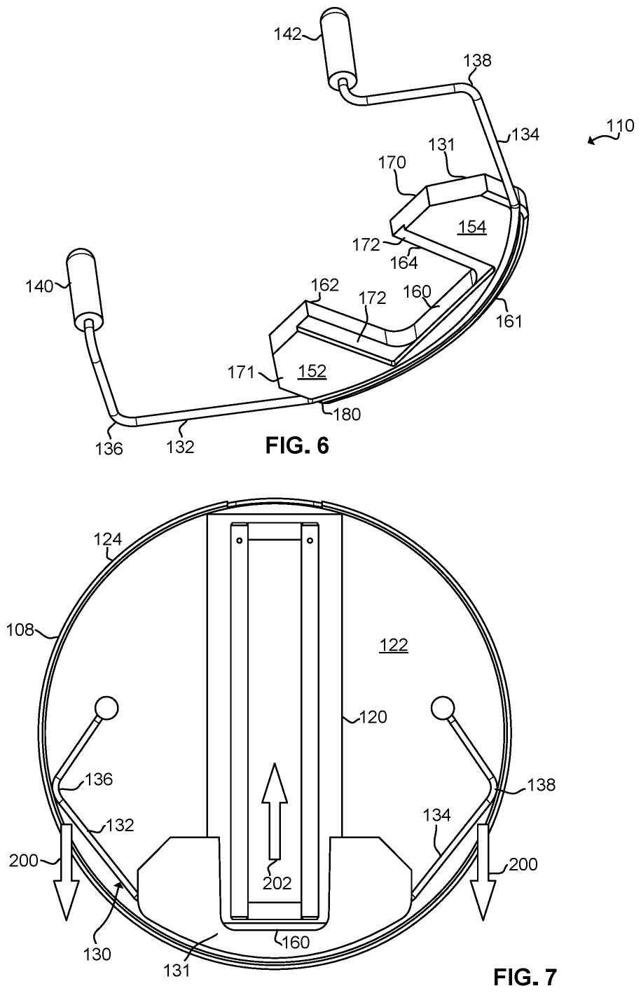

Referring now to FIGS. 5-6, the restraint device 110 can be removable from the petri dish 108 (and slidably disengaged from the microslide 120). In this embodiment, the housing 131 can include a curved rear surface 161 that extends between the first side 152 and the second side 154 across a rear face of the base 150 opposite from the recess 156. The curved surface 161 may have a radius of curvature (or other curved profile) that mates with of the interior curvature of the wall 124 of the petri dish 108.

The housing recess 156 is formed in the housing 131 and has a round-cornered rectangular shape that is dimensioned to slidably receive a first end of a microslide (e.g., the microslide 120). The housing recess 156 is formed by a base recess wall 160, a right recess wall 162, and a left recess wall 164. The housing recess can have an open end between the right recess wall 162 and the left recess wall 164, the open end can be aligned with the base recess wall 160.

As depicted in FIG. 6, the base recess wall 160, the right recess wall 162, and the left recess wall 164 each connect to a top surface 170 of the housing 131. The housing 131 can include a recessed area 172 that is dimensioned to receive the microslide 120 and hold a top surface of the microslide 120. The recessed area 172 can be recessed into a bottom surface 171 of the housing 131. The recessed area 172 can extend outwardly from each of the right recess wall 162 and the left recess wall 164 between a bottom of the right recess wall 162 and the left recess wall 164 and the bottom surface 171 of the housing. The recessed area 172 can be aligned with the base recess wall 160, and the base recess wall 160 with the recessed area 172 can be dimensioned to slidably engage with a microslide (e.g., the microslide 120) along four sides of the microslide when the microslide is retained by the restraint device 110. For example, the housing 131 can contact a top surface, an end surface, and two side surfaces of the microslide. The recessed area 172 can receive the microslide such that the right recess wall 162 and the left recess wall 164 extend over a portion of the top surface of the microslide. The right recess wall 162 and the left recess wall 164 can therefore assist in keeping the microslide in a desired position.

In this embodiment, the housing 131 has a slot 180 that is formed into the bottom surface 171 of the housing 131. The slot 180 can follow the curved surface 161 of the housing 131, and the slot 180 can extend from the right side 152 to the left side 154 and across the base 150. The slot 180 can have a depth that is greater than or equal to an outer diameter of the first arm 132 to receive the spring bias component 130 in the slot 180. In some aspects, the slot 180 can connect the housing 131 to the spring bias component 130 by a friction fit between the slot 180 and the portion of the spring bias component 130 that is received in the slot 180. In some aspects, the slot 180 can extend around a perimeter of the housing 131 along a back surface of the base 150.

Referring now to FIG. 7, when the restraint device 110 is seated within the petri dish 108 and slidably mated with the microslide 120, the restraint device 110 generates one or more forces that urge the microslide 120 into the operative position within the petri dish 108. The spring bias component 130 engages against the wall 124 of the petri dish to generate extension forces 200 on each side of the restraint device 110 with at least a directional force component a first direction. For example, the extension forces 200 can be generated by the restraint device 110 in response to the first and second arms 132 and 134 urging the elbows of the restraint device (e.g., the first elbow 136 and the second elbow 138) outwardly away from one another and against the wall 124 of the petri dish 108. The first elbow 136 and the second elbow 138 remain in contact against the wall 124, and the arms 132 and 134 translate the reactionary force to the housing 131.

As detailed above, the housing 131 is engaged with the microslide 120 (in the recess 156), and as such, the restraint device 110 urges the housing 131 to apply a retainer force 202 against the microslide 120 in response to the extension forces 200 (from the elbows 136 and 138 being urged against the wall 124). The retainer force 202 can be in opposite second direction that is different, or opposite from, at least the component of the extension forces 200 in the first direction (detailed above). The retainer force can push against a first end of the microslide 120 so that a second end of the microslide 120 is urged against the portion of the wall 124 positioned opposite from the first end of the microslide 120. Accordingly, in some implementations, the contact between the wall 124 and each respective elbow 136 and 138 compresses the arms 132 and 134 at least partially toward one another and generates a bias force (e.g., retainer force 202) that urges the microslide 120 into an operative position between the housing 131 and a portion of the wall 124. In doing so, the microslide 120 is retained in a fixed position in the petri dish 108 during use (e.g., during movement and engagement with a microscope or other instruments) while also providing simple release and removal of the microslide 120 (and, optionally, the restraint device 110) from the petri dish 108.

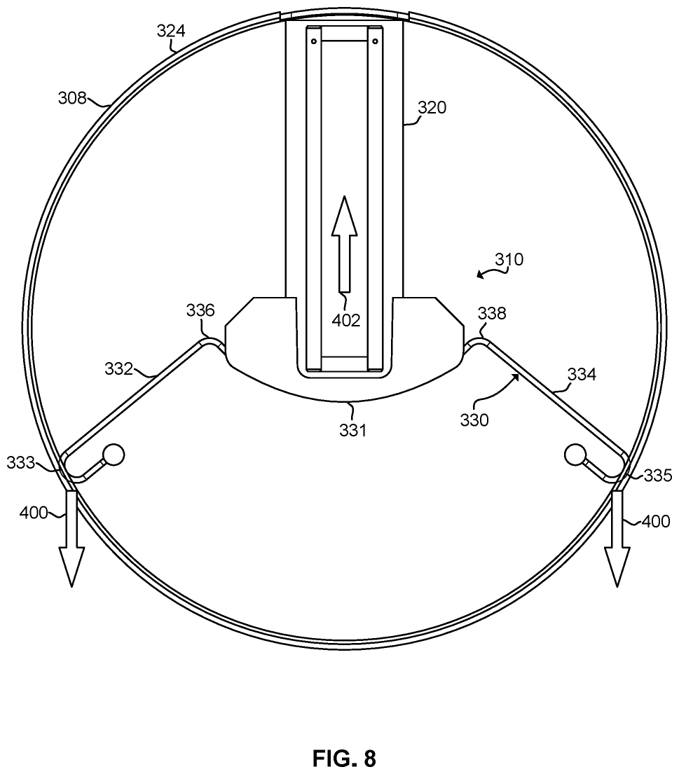

Referring now to FIG. 8, in another example of a restraint device 310 configured to releasably secure a microslide 320 in a petri dish 308, the restraint device 310 can include arms 332 and 334 that are shaped differently from the previously described arms 132 and 134. In some aspects, the petri dish 308 and the restraint device 310 can share features with the previously described petri dish 108 and the restraint device 110. The petri dish 308 and the restraint device 310 can differ from the previously described petri dish 108 and the restraint device 110 in other ways. For example, the petri dish 308 can have a larger diameter that is larger relative to the longitudinal length of the microslide 320 compared to relative sizing between the previously described petri dish 108 and microslide 120.

In this embodiment, the restraint device 310 includes a spring bias component 330 and an housing 331 that can share the many features of the spring bias component 130 and the housing 131.

For example, the spring bias component 330 can include one or more arms including a first arm 332 and a second arm 334. The first arm 332 and the second arm 334 can be flexible or semi flexible arms that extend from opposite sides of the housing 331. The first arm 332 can include a first elbow 336, and the second arm 334 can include a second elbow 338. Additionally, the spring bias component 330 can differ from the spring bias component 130 in certain instances. For example, the first arm 332 and the second arm 334 can include a first housing elbow 333 (spaced apart from the first elbow 336) and a second housing elbow 335 (spaced apart from the second elbow 338), respectively. The first arm 332 extends away from the housing 331 toward the first elbow 336, which bends the arm 332 in a direction toward the wall 324 and to the first housing elbow 333, which engages against the wall 324 and bends the arm 332 toward a free end (optionally having a handle at the free end). Similarly, the second arm 334 extends away from the housing 331 (oppositely from the first arm) and toward the second elbow 336, which bends the arm 334 in a direction toward the wall 324 and toward the second housing elbow 335, which engages against the wall 324 (opposite from the portion of the wall 324 engaged by elbow 333) and bends the arm 334 toward a free end (optionally having a handle at the free end).

The flexible first arm 332 and second arm 334 can be elastically deformable such that a user can flex the first arm 332 and the second arm 334 about the first elbow 336 and the second elbow 338, respectively. Similar to previously described embodiments, the spring bias component 330 can include a spring wire structure or other elastic materials to facilitate the elastic deformation of the spring bias component 330.

The restraint device 310 can generate one or more forces to retain the microslide 320 in an operative position within the petri dish 308. For example, the arms 332 and 334 of spring bias component 330 are biased to urge the first housing elbow 333 and the second housing elbow 335 outwardly away from one another and against the wall 324 of the petri dish 308, which generates extension forces 400 on each side of the restraint device 310 with at least a directional force component a first direction. For example, the extension forces 400 can be generated by the restraint device 310 in response to the first and second arms 332 and 334 urging the first and second housing elbows 333 and 335 outwardly away from one another and against the wall 324 of the petri dish 308. The first housing elbow 333 and the second housing elbow 335 remain in contact against the wall 324, and the arms 332 and 334 translate the reactionary force to the housing 331. As depicted in FIG. 8, the housing 331 can be releasably engaged with the microslide 320 (in the recess), and as such, the restraint device 310 urges the housing 331 to apply a retainer force 402 against the microslide 320 in response to the extension forces 400 (from the elbows 333 and 335 being urged against the wall 324). The retainer force 402 can be in a second direction that is different, or opposite from, at least the directional component of the extension forces 400 in the first direction (detailed above). The retainer force can push against a first end of the microslide 320 so that a second end of the microslide 320 is urged against the portion of the wall 324 positioned opposite from the first end of the microslide 320. Accordingly, in some implementations, the microslide 320 is retained in a fixed position in the petri dish 308 during use (e.g., during movement and engagement with a microscope or other instruments) while also providing simple release and removal of the microslide 320 (and, optionally, the restraint device 310) from the petri dish 308.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

Accordingly, other implementations are within the scope of the following claims.

What is claimed is:

1. A restraint device for retaining a slide instrument in a petri dish, comprising:
a spring bias component having one or more arms, each arm having one or more elbows; and
a housing connected to the spring bias component, the housing having a base that defines a recess to releasably mate with at least a portion of a slide instrument;
wherein the one or more arms of spring bias component are shaped to elastically deform in response to engagement with a wall of a petri dish so that the spring bias component generates a retainer force applied from the base and toward the recess.

2. The restraint device of claim 1, wherein a portion of the spring bias component is integrated in the base of the housing.

3. The restraint device of claim 2, wherein the base of the housing defines a slot that receives a portion of the spring bias component to connect the spring bias component to the housing.

4. The restraint device of claim 1, wherein the recess of the base extends longitudinally away from a rear face of the base and is configured to slidably receive the slide instrument at a front face of the base.

5. The restraint device of claim 4, wherein the retainer force applied from the base is longitudinally toward the slide instrument when the slide instrument is releasably mated with the base.

6. The restraint device of claim 1, wherein the spring bias component has one or more handles that extend from a free end of each arm.

7. The restraint device of claim 1, wherein the spring bias component comprises a spring wire.

8. The restraint device of claim 1, wherein the spring bias component comprises an elastic material.

9. A system comprising:
a microslide having a major surface extending between a first end and second end and one or more electrodes;
a petri dish having a base and a curved sidewall that defines a cavity to receive the microslide; and
a restraint device comprising:
a spring bias component having one or more arms, each arm having one or more elbows; and
an housing connected to the spring bias component, the housing having a base that is releasably matable with a first end of the microslide;
wherein in response to contact between each elbow and a wall of the petri dish, the spring bias component generates a retainer force at the base to urge the microslide against the curved sidewall of the petri dish.

10. The restraint device of claim 9, wherein a portion of the spring bias component is integrated in the base of the housing.

11. The restraint device of claim 9, wherein the base of the housing defines a recess to releasably mate with the first end of the microslide.

12. The restraint device of claim 11, wherein the recess of the base extends longitudinally away from a rear face of the base and is configured to slidably receive the microslide at a front face of the base.

13. The system of claim 9, wherein the base of the housing has a slot that receives a portion of the spring bias component to connect the spring bias component to the housing.

14. The system of claim 9, wherein the restraint device is symmetric about a longitudinal plane that extends along a recess of the base.

15. The system of claim 9, wherein the spring bias component has one or more handles that extend from an end of each arm.

16. The system of claim 9, wherein the spring bias component comprises a spring wire.

17. The restraint device of claim 9, wherein the spring bias component comprises an elastic material.

18. A method for securing a microslide in a petri dish, the method comprising:
inserting a microslide in a petri dish;
elastically deforming a spring bias component of a restraint device to fit in the petri dish;
slidably mating a housing base of the restraint device with the microslide;
urging one or more arms of the spring bias component against a wall of the petri dish; and
in response to said urging, applying a retainer force from the housing base to the microslide to releasably secure the microslide in an operative position within the petri dish.

19. The method of claim 18, wherein the spring bias component comprises an elastic material.

20. The method of claim 18, a portion of the spring bias component is integrated in the housing base.
